(19) United States
(12) Patent Application Publication
(10) Pub. No.: US 2026/0306773 A1
(43) Pub. Date: Oct. 8, 2026

(54) NO delivery apparatus incorporating flexible conduit purge means

(71) Applicant: INOSYSTEMS, ANTONY (FR)

(72) Inventors: Frederic MARCHAL, ANTONY (FR); Nathan PROUVEZ, ANTONY (FR); Yann BLANDIN, ANTONY (FR); Thierry BOULANGER, ANTONY (FR)

(21) Appl. No.: 19/554,885

(22) Filed: Mar. 3, 2026

(30) Foreign Application Priority Data
Apr. 8, 2025 (FR) 2503723

(51) Int. Cl. A61M 16/00; A61M 16/08; A61M 16/20

(52) U.S. Cl. CPC A61M 16/024; A61M 16/0883; A61M 16/202; A61M 2202/0007; A61M 2202/0208; A61M 2202/0275; A61M 2205/3331; A61M 2205/505

(57) ABSTRACT

The invention relates to an NO delivery apparatus (1) for supplying an NO-containing gas comprising a main gas circuit (200) with an NO inlet (2) configured for the coupling of a flexible conduit (12) conveying the NO-containing gas. First flow control means (210) control the circulation of the flow of gas in the main gas circuit (200) to which there is connected a purge circuit (250) having a purge outlet (6) in communication with the atmosphere. Second flow control means (252) for the purge circuit (250) control the circulation of gas towards the purge outlet. Operating means (205) control the flow control means (210; 252) so as to halt the circulation of gas in the main gas circuit (200) and allow the circulation of gas in the purge circuit (250) in response to user actuation of purge initiating means (7) such as, for example, a virtual button displayed on a screen. Gas supply installation (100) comprising the apparatus (1).

### CROSS-REFERENCE TO RELATED APPLICATIONS

This patent application claims priority to French Patent Application No. 2503723, filed Apr. 8, 2025 and the entire contents of which are incorporated herein by reference.

### TECHNICAL FIELD

The invention relates to an apparatus for delivering NO and to an installation for supplying an NO-based gas mixture to a patient, comprising such an NO delivery apparatus, allowing the user to purge a flexible conduit connected to the main gas circuit of the NO delivery apparatus when the gas vessel supplying said flexible conduit is empty or almost empty and needs to be replaced with a full vessel.

### BACKGROUND

Inhaled nitric oxide (NO or iNO) is a gaseous medicament commonly used to treat patients suffering from acute pulmonary hypertension, in particular pulmonary vasoconstrictions in adults or children, including newborns (PPHN), as described for example in EP560928 or EP1516639.

To implement therapy by inhaled NO, use is made of a gas supply installation, also known as a gas or NO administration installation, comprising an NO delivery apparatus and a medical ventilator, that is to say a respiratory assistance apparatus, each supplying a patient circuit.

The NO delivery apparatus makes it possible to inject a gas mixture based on NO, typically an NO/nitrogen mixture, into the patient circuit, which is also supplied with a gaseous flow containing oxygen (at least approximately 20 vol %), such as air or an oxygen/nitrogen (O₂/N₂) mixture, supplied by the medical ventilator. The patient circuit generally comprises one or more flexible conduits which are fluidically connected to a respiratory interface, such as a tracheal intubation tube or the like, which is used to supply to the patient to be treated a therapeutic gas mixture containing a given quantity or dose of NO, that is to say a dosage, typically of between 5 and 40 ppmv of NO, or some other dosage chosen by the medical practitioner.

Gas supply installations of this type are described for example in EP3821929, EP4209243, EP4241817, EP4241812 and EP4295882.

The gaseous NO, typically a gaseous NO/nitrogen mixture, supplied to the NO delivery apparatus, generally comes from gas vessels, simply referred to as “NO cylinders”, fluidically connected to the apparatus by flexible hoses or conduits that serve to convey the gas.

Fluidically coupling the NO delivery apparatus to two NO (i.e. NO/nitrogen mixture) cylinders by means of suitable flexible hoses makes it possible to avoid any interruption to the treatment of the patient since when one NO cylinder is empty, the other NO cylinder which is full can then take over and supply the NO/N₂ mixture to the NO delivery apparatus.

Usually, the connection between each NO cylinder and the NO delivery apparatus is provided by a flexible hose or conduit or the like which is coupled to the outlet of an external pressure-reducing device, namely a gas pressure regulator, that is attached to the outlet coupling of the gas distribution valve with which the NO cylinder is fitted. The gas pressure regulator is able to reduce the pressure of the gas leaving the cylinder, which may be as high as 150 bar abs (when the cylinder is full), down to a given lower pressure, for example between 3 and 10 bar abs.

In order to replace an empty (or almost empty) NO cylinder with a full fresh NO cylinder, it is necessary to be able to disconnect the empty NO cylinder (i.e. the one that is to be replaced) from the flexible hose or conduit that is fluidically connected to the NO delivery apparatus, particularly from the gas pressure regulator to which the flexible conduit is attached.

Now, this cannot be done until the user has purged the residual gaseous NO (i.e. NO/N₂ mixture) pressure, which may be several bar, for example 3 to 7 bar, that is trapped in the flexible hose between the NO delivery apparatus and the NO cylinder and/or regulator.

In order to do this, the user needs first of all to disconnect the flexible hose or conduit at the NO delivery apparatus end, particularly from the regulator, then purge said flexible hose using a dedicated tool, in order to discharge the residual gaseous-NO pressure contained therein, so as then to be able to unscrew the regulator from the empty cylinder before refitting it to the replacement full NO cylinder.

It will be appreciated that such an operation is awkward, time-consuming and may present risks to the user on account of the residual pressure remaining in the flexible hose. Moreover, because the conduit is subjected to a residual internal pressure of several bar, for example 3 to 7 bar, it is very difficult, or even sometimes near-impossible, to remove the pressure regulator device.

EP4241817 and EP2522384 disclose the need to regularly purge the NO delivery apparatus, and U.S. Pat. No. 6,125,846 specifies that the purge may be automatic or manual, namely instigated by an operator. However, those documents do not detail the purge operation, or the operation of replacing an empty NO cylinder with a new, i.e. full, cylinder.

One problem is therefore that of how to simplify the operations of replacing an empty NO cylinder in order to avoid the user having to perform awkward disconnection and reconnection operations, and also manual purging of the flexible hose, when replacing an empty NO cylinder supplying an NO delivery apparatus that is preferably coupled to two (or more) NO cylinders, which apparatus forms part of an installation for supplying an NO-containing gas mixture to a patient.

### SUMMARY

One solution according to the invention concerns an NO delivery apparatus for supplying an NO-containing gas, typically an NO/N₂ gas mixture, comprising:
a main gas circuit for conveying the NO-containing gas between at least one NO inlet and at least one NO outlet,
said at least one NO inlet configured to allow the fluidic connection thereto of a flexible conduit so as to supply the main gas circuit with the NO-containing gas carried by said flexible conduit,
first flow-control means arranged on said main gas circuit and configured to control the circulation of the flow of NO-containing gas in said main gas circuit,
microprocessor-containing operating means,
a purge circuit fluidically coupled to the main gas circuit between said at least one NO inlet and the first flow control means, said purge circuit comprising a purge outlet in fluidic communication with the atmosphere,
second flow control means arranged on said purge circuit and configured to control the circulation of gas in said purge circuit towards the purge outlet, and
purge initiation means that can be actuated by the user.

According to the invention, the apparatus further comprises a display touchscreen, and:
the purge initiation means comprise a virtual purge-start button displayed on the display touchscreen, and
the operating means are configured to operate the first and second flow-control means in such a way as to halt (i.e. prevent) the circulation of gas in at least part of the main gas circuit and allow (i.e. permit) gas to circulate in the purge circuit in response to the user pressing a finger on (i.e. bringing a finger into contact with) the virtual button, i.e. the purge initiation means.

Depending on the embodiment considered, the apparatus according to the invention for delivering NO, i.e. an NO/N₂ gas mixture, may comprise one or more of the following features:
the first and second flow control means comprise at least one valve, particularly electrically-operated valves referred to as “main” operated by the operating means.
the display touchscreen comprises a touch tile or the like.
the virtual purge-start button is displayed on the display touchscreen inside a window displayed on the screen, i.e. a dedicated window, typically a start-purge window.
the purge-start button is configured to be activated by the user bringing a finger into contact with or pressing on a given zone of the touchscreen.
the touchscreen is configured to detect contact of the user's finger on the given zone of the touchscreen that corresponds to the purge-start button, namely when the purge-start button is or has been activated, and to transmit an activation signal to the operating means.
the operating means are configured to receive and process the activation signal.
the operating means are configured to interpret or recognise the activation signal as being a signal to begin the purge.
the operating means are configured to begin a purge in response to said begin-purge signal.
the operating means command a purge to begin by acting on the first and second flow control means in response to actuation of the purge initiation means, namely to the user pressing a finger (or another digit), onto the virtual button.
the main gas circuit comprises two inlet portions arranged in parallel.
each inlet portion comprises an NO inlet able to supply the main gas circuit with NO-containing gas, typically an NO/N₂ gas mixture.
the two inlet portions are fluidically coupled to one another at a junction point of the main gas circuit.
the purge circuit comprises two joining portions arranged in parallel.
each joining portion is coupled to one of the inlet portions of the main gas circuit, upstream of the first flow control means arranged on said main gas circuit.
the two joining portions are fluidically coupled to one another at a meeting point of the purge circuit.
second flow control means are arranged on the two joining portions, these typically being “purge” electrically-operated valves, likewise operated by the operating means.
in response to the user pressing (a finger) on the virtual purge-start button displayed on the display touchscreen, the operating means are configured to operate the main and purge electrically-operated valves in such a way as to:
   halt the circulation of gas in that part of the main gas circuit that is situated downstream of the main electrically-operated valves by closing, or keeping closed, the two main electrically-operated valves;
   keep one of the purge electrically-operated valves closed; and
   allow gas to circulate in the purge circuit by moving the other of the purge electrically-operated valves into the open position in order to allow the gas to circulate in the purge circuit.
it comprises two NO inlets configured each to be coupled to one flexible conduit for conveying gas, i.e. the NO/N₂ mixture.
the NO inlet or inlets may each be borne by a fluidic connector or coupling.

Moreover, depending on the embodiment considered, the apparatus according to the invention for delivering NO, i.e. an NO/N₂ gas mixture, may comprise one or more of the following additional features:
the NO vessels, i.e. gas cylinders, contain an NO/N₂ mixture containing between 100 and 5000 ppmv of NO, the remainder being nitrogen, preferably containing between 100 and 2000 ppmv, typically between 200 and 1000 ppmv.
it is supplied with a gaseous mixture formed of nitrogen and NO, i.e. a binary mixture.
a residual NO/N₂ mixture contained in the empty NO vessel(s) is at a preset pressure referred to as residual pressure, which is preferably less than or equal to 25 bar, for example of the order of 25 bar.
it comprises dose adjustment means which are configured to allow a user to fix or select an NO content setpoint corresponding to the desired final proportion of NO in the combined gaseous mixture, i.e. a dosage.
the dose adjustment means form part of an HMI (human-machine interface) or UGI (user graphics interface).
the screen is of the type with colour display.
the digital screen, i.e. touch tile screen, forms part of the HMI.
the dose adjustment means comprise one or more touch tiles, that can be actuated by the user, typically by touching or pressing them with a finger (or another digit).
the dose adjustment touch button(s) are displayed on the digital screen.
the NO content setpoint is between 1 and 80 ppmv, typically between 5 and 40 ppmv.
it comprises storage means namely a computer memory, for example a flash memory, RAM or the like.
the operating means comprise a (micro)controller or the like.
the operating means comprise one or more (micro)processor(s) arranged on one or more electronic boards.
the operating means comprise one or more (micro)processor(s) which implement one or more algorithm(s), in particular one or more algorithm(s) for operating or controlling valves, for processing flow rate or pressure measurements, etc.
the storage means are arranged on the electronic board.
it is supplied with electric power by one or more electric current sources, typically the mains supply (110/220 V) and/or one or more rechargeable batteries.
it comprises a peripheral shell or casing.
the two inlet portions, the main gas circuit, the purge circuit, the first and/or second flow control means are arranged in the peripheral shell or casing of the apparatus.

The invention also relates to an installation for supplying an NO-containing gas mixture to a patient, comprising:
at least one NO vessel containing the NO-containing gas, preferably two vessels for NO, typically an NO/N₂ gas mixture,
the NO delivery apparatus according to the invention, and
at least one flexible conduit fluidically connecting said at least one NO vessel to the NO delivery apparatus, each NO vessel preferably being connected to the NO delivery apparatus by a flexible conduit.

Depending on the embodiment in question, the gas supply installation according to the invention may comprise one or more of the following features:
at least one pressure-reducing device, such as a gas pressure regulator, is arranged downstream of said at least one NO vessel.
said at least one flexible conduit is fluidically tuned to said at least one pressure-reducing device.
it further comprises a medical ventilator configured to supply a flow of respiratory gas containing O₂ (≥20% vol.), s u ch a s air or an O₂/N₂ mixture.
it further comprises a respiratory circuit to which the medical ventilator and the NO delivery apparatus are fluidically connected, particularly to an inspiratory branch of said respiratory circuit.
a flow rate sensor is arranged in the respiratory circuit.
an injection device enables the flow of NO-containing gas (e.g. an NO/N₂ mixture) to be injected into the flow of respiratory gas containing O₂ (e.g. air or an O₂/N₂ mixture) so as to obtain a combined gas mixture, i.e. a final mixture, containing NO, oxygen and nitrogen.
the medical ventilator delivers a respiratory gas containing at least about 20% by volume of oxygen, preferably at least about 21% by volume of oxygen.
the medical ventilator comprises a motorized blower (i.e. turbine, compressor or similar) delivering the respiratory gas, typically air or an oxygen/nitrogen mixture or, according to another embodiment, an internal gas circuit comprising one or more proportional valves for conveying the gas and controlling its supply, in particular its flow rate. Such a ventilator is generally supplied with respiratory gas via one or more wall outlets supplied with gas from a network of pipes in a hospital establishment or building, typically with air or an oxygen/nitrogen mixture.
the medical ventilator comprises control means or a control device, such as one or more electronic control boards. Preferably, the control means of the medical ventilator operate or control the motorized blower or, depending on the circumstances, the proportional valves of the medical ventilator.
the medical ventilator is of HFO type or comprises an HFO function, that is to say it is able to produce high-frequency oscillations.
each NO vessel contains an NO/N₂ gas mixture containing between 100 and 2000 ppmv of NO, the remainder being nitrogen (N₂), preferably between 100 and 1000 ppmv of NO, and for example 450 or 800 ppmv.
each NO vessel contains an NO/N₂ gas mixture conditioned at a pressure (measured before start of withdrawal) of between 10 and 250 bar, preferably of more than 50 bar, typically of more than 100 bar.
each NO vessel is or comprises one or more gas cylinders with a capacity of between 0.5 and 50 L (water equivalent).
each NO vessel comprises a cylindrical body made of steel or of aluminium alloy.
each NO vessel is equipped with what is called a “simple” gas distribution valve (without an integrated pressure regulator) or with an “integrated pressure regulator” (IGPR) valve incorporating expansion (i.e. pressure reduction) means, preferably with an IGPR valve.
each gas distribution valve (i.e. simple or IGPR type) is protected by a protective cap, for example made of metal or polymer.
each NO vessel is equipped with a “simple” valve (i.e. without an integrated pressure regulator), and a gas pressure regulator device is arranged downstream of each valve.
the pressure-reducing means, i.e. the gas pressure regulator device, are arranged downstream of the “simple” valve of each NO container containing the NO-based gas, typically an NO/N₂ mixture, so as to decrease or reduce the pressure of the NO-based gas, typically an NO/N₂ mixture, leaving the valve in question, i.e. each valve, and thus obtain the gas at the desired expansion pressure (PD) of less than 10 bar.
the pressure regulator device is fixed to a (simple) valve by being fluidically connected to the pressure outlet of said valve, for example by screwing or the like.
the pressure-reducing means are configured to reduce the pressure of the NO-based gas, typically an NO/N₂ mixture, to an expansion pressure of less than or equal to 10 bar, preferably less than 8 bar, typically between 2 and 7 bar, for example between 5 and 6 bar.
the or each flexible conduit is configured to convey the NO-based gas after its pressure has been reduced to the expansion pressure of less than or equal to 10 bar, i.e. a pressure-regulated gas.
the or each flexible conduit comprises a polymer hose.
the respiratory circuit comprises an inspiratory branch and an expiratory branch, typically flexible conduits forming the inspiratory branch and the expiratory branch, for example hoses made of polymer.
the inspiratory branch and the expiratory branch, e.g. flexible conduits, are connected at a joining piece, such as a Y-piece.
the inspiratory branch and/or the expiratory branch are fluidically connected to a patient respiratory interface, preferably via the joining piece.
the patient respiratory interface comprises a tracheal intubation tube or a breathing mask or the like.
the inspiratory branch and the expiratory branch are moreover fluidically connected to outlet and inlet orifices, respectively, of the medical ventilator.
the respiratory circuit, in particular the inspiratory branch, may comprise a gas humidifier.
the gas humidifier is arranged downstream of the injection device in such a way as to be able to humidify the gas before the latter is administered by inhalation to the patient.
the medical ventilator is supplied electrically by one or more electric current sources, typically the mains supply (110/220 V) and/or one or more rechargeable batteries.
the NO vessel(s) i.e. the NO cylinder(s) and/or the NO delivery apparatus are arranged on a wheeled transport cart.

According to another aspect, the invention also relates to a method for therapeutic treatment of a person, i.e. a human patient (i.e. adult, child, adolescent or neonate), suffering from pulmonary hypertension and/or hypoxia, which cause pulmonary vasoconstrictions or similar, said method comprising administration by inhalation, to the person requiring it, of a gaseous mixture containing from 1 to 80 ppmv of NO and at least 20 vol % of oxygen approximately, preferably at least 21 vol % of oxygen approximately, by means of a gas delivery installation, as described above according to the invention, comprising an NO delivery apparatus for delivering NO at the desired dosage, so as to treat (at least partially) said pulmonary hypertension and/or said hypoxia, which can be caused by one or more pulmonary diseases or disorders, typically PPHN (persistent pulmonary hypertension of the newborn) or ARDS (acute respiratory distress syndrome), or can be caused by heart surgery with placement of the patient on extracorporeal blood circulation (ECC).

### DEFINITIONS

In general, within the context of the invention:
“ppmv” means parts per million by volume,
“vol%” means percentage by volume.
“NO” denotes nitric oxide.
“NO₂” denotes nitrogen dioxide.
“N₂” denotes nitrogen.
“O₂” denotes oxygen.
the pressures are expressed in bar absolute, abbreviated to “bar”.
the terms “concentration”, “quantity”, “proportion”, “dose” and “content” are considered to be equivalent and interchangeable.
the terms “conduit”, “hose”, or “line” are considered to be equivalent and interchangeable.
the terms “means of/to/for” are considered to be wholly equivalent to and capable of being substituted by the terms “device of/to/for”, for example the term “control means” may be replaced by “control device”, the term “valve means” may be replaced by “valve device”, the “storage means” may be replaced by “storage device”, etc.
a “pressure measurement” means a pressure value (e.g. a numerical value) or a signal representative of such a pressure value, which reflects or corresponds to the gaseous pressure measured by a pressure sensor or the like.
the terms “upstream” and “downstream” are used in relation to the normal direction of gas flow, i.e. in the direction from the gas vessels to the delivery apparatus and then to the patient.
an NO vessel, such as a gas cylinder, is said to be “empty” or “almost empty” when it contains a small quantity or pressure of NO-based gas because most of the gas that it contained has been consumed/used up, for example if the vessel contains a quantity of gas corresponding to a preset gas pressure, for example less than or equal to 25 bar of NO-based gas.

### BRIEF DESCRIPTION OF VIEWS OF DRAWINGS

The invention will now be understood more clearly from the following detailed description, provided by way of non-limiting illustration, with reference to the appended figures, in which:

FIG. 1 shows schematically one embodiment of a gas administration installation according to the invention.

FIG. 2 shows schematically one embodiment of part of the internal architecture of the NO delivery apparatus of a gas administration installation according to the invention, such as that of FIG. 1.

### DETAILED DESCRIPTION

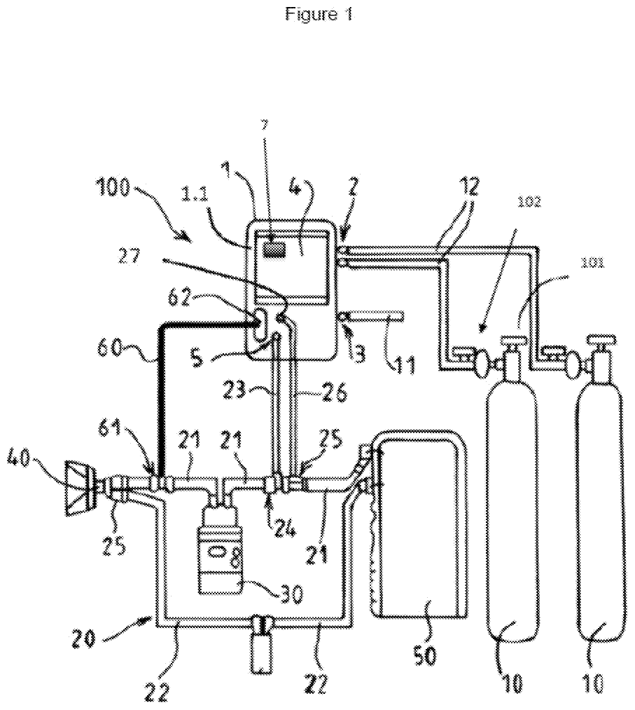

FIG. 1 shows schematically one embodiment of a gas administration installation 100 according to the invention, comprising an NO supply apparatus 1 which supplies a gaseous mixture based on nitric oxide (NO), and a medical ventilator 50 which supplies a gas containing at least 20 vol% of oxygen, such as air or the like.

The installation 100 comprises NO sources, namely in this case two pressurized gas cylinders or vessels 10 each containing an NO-based gas, namely an NO/N₂ gas mixture, namely in this case an NO/N₂ gas mixture containing between 100 and 5000 ppmv of NO (remainder N₂), preferably between 100 and 1000 ppmv of NO (remainder N₂), for example 450 or 800 ppmv of NO (remainder N₂), or any other suitable concentration, which supply an NO/N₂ mixture to the device or apparatus 1 for delivering or supplying NO, making it possible to monitor and control the supply of the NO/N₂ gas mixture.

The NO/N₂ gas mixture is generally conditioned in each vessel 10 at a pressure, referred to as “high pressure”, of at least 150 bar, which corresponds to the initial pressure prevailing in each vessel before any withdrawal begins, that is to say measured before any withdrawal or any use of gas (i.e. when the vessel is full). In some cases, it can be as much as 230 to 250 bar, or even more. Of course, the pressure of the gas in the vessels 10 decreases as the gas is withdrawn, that is to say as the gas is progressively used, thus causing progressive emptying of the vessels 10.

As is illustrated in FIG. 1, each vessel 10 comprises a body of generally cylindrical or ogive shape, containing the gas within its internal volume, which body is surmounted by a gas distribution valve 101 serving to control the exit of the gas from the internal volume of the body of the vessel 10.

Each valve 101 is what is called a “simple” valve, that is to say one that does not contain internal pressure regulating means. As a result, external pressure-reducing means, namely a pressure-reducing device 102, also referred to as a gas pressure regulator, are arranged downstream of each valve 101 in order to ensure a reduction of the gas pressure to a desired expansion pressure (PD) which is adjusted at each pressure regulator, typically an expansion pressure of less than or equal to 10 bar, preferably of less than or equal to 8 bar, typically of between 4 and 7 bar, advantageously of between 5 and 6 bar. Each pressure-reducing device 102 is for example screwed onto the pressure outlet of the valve 101 on which it is arranged, which is to say that it is mounted on the pressure outlet coupling of the regulator 102.

Each gas cylinder 10 is fluidically connected to the NO supply apparatus 1 via a flexible conduit 12, namely a gas supply line, typically flexible hoses or the like, which may be fitted with gas pressure monitoring devices, notably one or more pressure gauges or the like. The upstream end of the flexible conduit 12 is coupled, as illustrated in FIG. 1, to the outlet of the pressure-reducing device 102.

Alternatively, according to another embodiment (not shown), the valves 101 could be of the type with an integrated gas pressure regulator (IGPR), that is to say the expansion means 102 would then be arranged within (i.e. integrated into) the body of each valve 101, for example an expansion valve cooperating with a valve seat. In that case, the gas leaving the valve 101 is at the desired expansion pressure, i.e. an expansion pressure of less than or equal to 10 bar, as before, and the flexible conduit(s) 12 then connects directly to the pressure outlet of said valve 101.

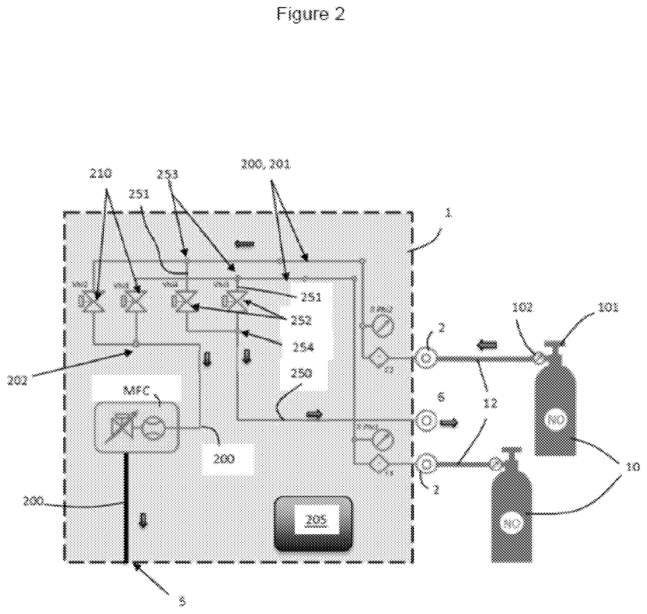

Each flexible conduit 12 carries the NO-based gas at the expansion pressure (PD), namely the expanded gas, to the apparatus 1. It connects fluidically to a gas inlet of the apparatus 1, referred to as NO inlet 2, which supplies the internal main gas circuit 200 of the apparatus 1, as detailed in FIG. 2, that serves to carry the NO-based gas within the NO supply apparatus 1, namely within the peripheral casing or shell 1.1 of the apparatus 1 between each NO inlet 2 and one (or more) NO outlet(s) 5.

Each NO inlet 2 is configured to allow the downstream end of a flexible conduit 12 to be fluidically coupled to it. For that purpose it is possible to use conventional fluidic-connection means, such as a fluidic coupling, a fluidic connector or the like, to which the flexible conduit 12 is attached for example by push-fitting.

In the embodiment of FIG. 2, the main gas circuit 200 comprises two inlet portions 201, arranged in parallel with one another, and two NO inlets 2, each inlet portion 201 being fluidically connected to one of the NO inlets 2 supplied with NO/N₂ mixture by a flexible conduit 12 carrying the gas (i.e. NO/N₂) from one or the other of the gas vessels 10. The two inlet portions 201 of the main gas circuit 200 are fluidically coupled to one another at a junction point 202.

The main gas circuit 200, particularly each inlet portion 201, is equipped with first flow control means 210, typically with control valves Vhi1, Vhi2 which are operated by operating means of the apparatus 1 so as to control the flow of gas within the main gas circuit 200, particularly within each inlet portion 201. The operating means 205 therefore act on the flow control means 210, typically on the control valves Vhi1, Vhi2.

The first flow control means 210 are therefore arranged between each NO inlet 2 and the junction point 202 at which the two portions 201 join one another As a preference, the operated control valves Vhi1, Vhi2 are electrically-operated valves, referred to as main electrically-operated valves Vhi1, Vhi2 or analogous operated valves.

More specifically, the operating means 205 may operate the control valves Vhi1, Vhi2, namely the main electrically-operated valves, to make them move into the open position and thus allow gas to pass through one of the valves or the other or, conversely, into the closed position, and thus prohibit any passage of gas through the other of the valves, and vice versa. The operating means may operate the control valves Vhi1, Vhi2 such that they are both in the closed position so as to prevent, i.e. prohibit or halt, any circulation of gas in the rest of the main circuit 200, namely notably downstream of the junction point 202.

In general, when one or the other of the control valves Vhi1, Vhi2 is in the open position, the circulation of the NO-based gas in the main gas circuit 200 is in the direction from the NO inlets 2 towards the first flow control means 210, i.e. the control valves Vhi1, Vhi2.

Usually, the operating means 205 of the apparatus 1 commanding the operated control valves Vhi1, Vhi2 comprise one (or more) microprocessor(s) implementing one or more algorithm(s) or other computer programs. The microprocessors are carried on an electronic board. The electronic board may also comprise storage means, such as a flash memory or the like.

The operating means 205 are electrically connected, via electrical connections or the like, such as electric cables, to the first flow control means 210, typically to the operated control valves Vhi1, Vhi2.

The gas pressure prevailing in the circuit 200, particularly in the two inlet portions 201, corresponds to the gas pressure carried by the flexible conduits 12, and therefore to the expansion pressure. This pressure of the gas within the two inlet portions 201 is continuously monitored, i.e. measured, by pressure-measuring means Phi1, Phi2, such as pressure sensors, arranged on these portions 201 and able to take pressure measurements within the portions 201 of the circuit 200 and supply these measurements to the operating means that process them.

Advantageously, the gas flow entering the circuit 200, particularly entering the two inlet portions 201, may be filtered by filtering means F1, F2, such as HEPA filters or the like, arranged in the two inlet portions 201, near the NO inlets 2.

Moreover, the NO delivery apparatus 1 of the installation 100 also comprises an oxygen inlet 3, which is fluidically connected, via an oxygen supply conduit 11 such as a flexible hose or the like, to an oxygen source (not shown), for example a pressurized oxygen cylinder or a hospital network, that is to say an oxygen supply line arranged in a hospital building. This makes it possible to feed the internal gas circuit 200 with oxygen when necessary.

The gas administration installation 100 of FIG. 1 further comprises a medical ventilator 50, that is to say a respiratory assistance apparatus, which supplies a flow of oxygen-based respiratory gas, that is to say one containing at least about 20 vol % of oxygen, preferably at least about 21 vol % of oxygen, such as air or an oxygen/nitrogen (N₂/O₂) mixture.

The medical ventilator 50 and the NO delivery apparatus 1 of the installation 100 are in fluidic communication with a respiratory circuit 20, also called the patient circuit, in particular with the inspiratory branch 21 of the circuit 20, which serves to convey the gas flow to the respiratory interface 40 supplying the therapeutic gas flow to the patient, that is to say a final or combined gas mixture containing the desired dosage of NO. The combined gas mixture to be administered to the patient is formed by mixing the oxygen-based gas flow (e.g. air or O₂/N₂ mixture) coming from the medical ventilator 50 and the NO-containing flow, typically an NO/N₂ gas mixture, delivered by the NO delivery apparatus 1.

For this purpose, the NO delivery apparatus 1 supplies or injects the NO/N₂ mixture into the inspiratory branch 21 of the circuit 20, via an injection conduit 23, which fluidically connects the main internal gas circuit 200 of the NO delivery apparatus 1 to an injection device 24 arranged on the inspiratory branch 21.

The injection device 24 is configured to mix the NO-containing gas from the NO delivery apparatus 1 with the flow of respiratory gas coming from the ventilator 50 and to obtain a combined gas mixture containing NO and oxygen, which is then administered to the patient.

The combined gas mixture contains NO at the desired dosage, nitrogen (N₂) and oxygen (O₂), namely an NO/N₂/O₂ gas mixture. Unavoidable impurities, e.g. argon, CO₂, NO₂, etc., may potentially be present.

The inspiratory branch 21 of the circuit 20 further comprises a gas humidifier 30 arranged downstream from the injection device 24, making it possible to humidify the combined NO/N₂/O₂ gas flow before it is administered by inhalation to the patient to be treated, by means of the respiratory interface 40, such as a tracheal intubation tube, a breathing mask or the like.

The circuit 20 comprises an expiratory branch 22 fluidically connected to the inspiratory branch 21 and to the respiratory interface 40 via a joining piece 25, typically a Y-piece.

Furthermore, a flow rate sensor 25, for example of the hot wire or pressure differential or mass flow type, is arranged on the inspiratory branch 21, between the ventilator 50 and the injection device 24. The flow rate sensor 25 is connected to a connection port 27 of the NO delivery apparatus 1 via one or more flow rate measurement lines 26, which are connected to said sensor-connection port 27, namely one or more electric cables or the like. The flow rate sensor 25 is used to measure the flow rate of gas delivered by the ventilator 50, such as air or N₂/O₂, circulating in the inspiratory branch 21, upstream from the injection device. These flow rate measurements taken by the flow rate sensor 25 make it possible more effectively to monitor or regulate the delivery of the flow of NO (i.e. N₂/O₂) by the NO delivery apparatus 1, particularly the NO flow rate. The flow rate measurements taken by the flow rate sensor 25 are processed by the (micro)processor operating means 205 of the NO delivery apparatus 1, which is typically a (micro)controller.

As schematically indicated in FIG. 2, in normal operation, the flow of NO/N₂ passes along the main gas circuit 200 of the apparatus 1, notably by a mass flow controller (or MFC) arranged downstream of the junction point 202, before leaving the main gas circuit 200 via the NO outlet 5 of the apparatus 1. As a preference, the MFC comprises a proportional electrically-operated valve and an additional flow rate sensor collaborating with the operating means 205.

The operating means 205 notably make it possible to adjust or control the flow rate of NO-based gas by operating the MFC, and particularly the proportional electrically-operated valve. In particular, during operation, they make it possible to determine the NO flow rate to be supplied in order to obtain the desired NO content in the combined mixture, namely the desired dosage of NO, particularly on the basis of the NO content setpoint regulated and/or fixed by the user, the composition of the NO/N₂ gas mixture coming from the gas cylinders 10, and one or more flow rate measurements taken by the flow rate sensor 25 arranged on the inspiratory branch 21.

In general, the operating means 205 also make it possible to process measurements, carry out calculations and/or to control or command all of the electromechanical elements of the apparatus 1, such as electrically-operated valves, displays, etc.

The internal gas circuit 200 of the NO delivery apparatus 1 can also comprise other elements or components, in particular one (or more) pressure sensor(s), one (or more) additional flow sensor(s) or flow meter(s), and/or calibrated-orifice devices or the like, and preferably a bypass or backup line enabling the MFC to be bypassed if it malfunctions for example or if there is a break in the signal coming from the flow rate sensor 25 arranged on the inspiratory branch 21.

The NO supply apparatus 1 also comprises a graphical user interface (or GUI) comprising a graphical display 4, preferably a touchscreen, i.e. one with a touch panel, serving to display various pieces of information or data, icons, curves, alerts, etc., and also virtual selection buttons and/or panes or windows, in particular for making choices, selections or for validating or cancelling choices or else entering information, such as desired values (e.g. flow rate, dosage of NO, etc.), or any other information or data useful to the healthcare personnel.

The display is preferably in colour, but it can also be in black and white.

The electrical power for the NO supply apparatus 1, in particular for the components requiring electrical current in order to operate, such as the operating means 205, the graphical display 4, etc., is provided conventionally by an electrical current source and/or electrical supply means (not shown), for example a connection to the mains (110/220 V) current, such as an electrical cord and connection socket, and/or one or more electric, preferably rechargeable, batteries, and/or a current transformer. The electrical power supply to the medical ventilator 50 is ensured in a similar manner, in particular by a connection to the mains current or by an internal battery.

Furthermore, the installation 100 also comprises a gas sampling line 60 which fluidically connects the inspiratory branch 21 (at 61) to an inlet port 62 of the NO supply device 1, such as a flexible hose or the like. This makes it possible to take samples of gas and send them to the NO supply device 1 where they are analysed in an internal gas analyser (not depicted), namely one contained within a calibration line comprising sensors for NO₂, for NO and for O₂, notably electrochemical cells, electrically connected to the operating means 205 so as to ensure that the composition of the final gas conforms to that of the desired NO/N₂/O₂ gas mixture, notably that it does not contain excessive amounts of toxic NO₂ species, that its oxygen content is not hypoxic, and that its NO content corresponds to the desired dosage, i.e. the dose of NO to be administered. The operating means 205 collect and process the signals from the sensors and can act in response to these signals, notably to calibrate the sensors or to trigger an alarm in the event of non-compliance.

As the patient's treatment progresses, each NO vessel 10, i.e. NO cylinder, empties and the pressure prevailing therein therefore decreases progressively until the vessel is empty or almost empty, namely until it contains a small quantity of NO-based gas because most of the gas that it contained has been consumed/used up during the treatment of the patient, typically when the NO vessel contains a quantity of gas corresponding to a preset gas pressure, referred to as “residual” pressure, for example less than 25 bar of residual NO-based gas. Of course, depending on the embodiment, the preset “residual” gas pressure, i.e. the residual-pressure threshold, may have some other value, namely may be greater than 25 bar or less than 25 bar.

In the embodiment proposed in the figures, the preset gas pressure referred to as the “residual” pressure is considered to be equal to around 25 bar.

As already explained, in order to replace an empty NO cylinder 10 with a fresh full NO cylinder 10, namely a “new” cylinder, it is necessary to be able to disconnect the NO cylinder 10 that is empty from the flexible conduit 12 that fluidically connects it to the NO delivery apparatus 1 and more specifically to disconnect the flexible conduit 12 from the gas pressure regulator 102, attached to the valve 101 of the empty cylinder 10 that is to be changed, to which regulator the flexible conduit 12 is to be attached.

Now, that cannot be done until the user has purged the residual pressure, representing a number of bars, for example 2 to 7 bar prevailing in the flexible hose 12 between the apparatus 1 and the regulator 102 of the NO cylinder 10 that is to be replaced.

In order to do that, in order to avoid the problems and disadvantages encountered in installations of the prior art, according to the present invention, the NO delivery apparatus is further equipped with a purge circuit 250 that is fluidically coupled (at 253) to the main gas circuit 200, particularly to the two portions 201, between the NO inlet(s) 2 and the first flow control means 210.

The purge circuit 250 comprising two joining portions 251 fluidically coupled (at 253) to the two inlet portions 201 of the main gas circuit 200 and also a purge outlet 6 in fluidic communication with the ambient atmosphere, namely with the air in the room.

The two joining portions 251 meet at a meeting point 254 situated on the purge circuit 250 upstream of the purge outlet 6.

Moreover, second flow control means 252 are arranged on said purge circuit 250, particularly on the two joining portions 251 of the purge circuit 250, upstream of the meeting point 254. These second flow control means 252 make it possible to control the circulation of gas in said purge circuit 250 towards the purge outlet 6, typically in the two joining portions 251 which are arranged in parallel with one another.

As before, the second flow control means 252 typically comprise control valves Vhi3, Vhi4 which are operated by the operating means 205 of the apparatus 1 so as to control the flow of gas within the purge circuit 250, particularly within each inlet portion 201. The operating means 205 therefore act on the second flow control means 252, namely on the control valves Vhi3, Vhi4, to authorise or prohibit/halt the circulation of gas. Once again, the control valves Vhi3, Vhi4 are preferably electrically-operated valves, referred to as purge electrically-operated valves, or similar operated valves, and are operated by the operating means 205 to make them move into the open position and thus allow the gas to pass through one of the valves or the other or, conversely, into the closed position, and thus prohibit any passage of gas through the other of the valves, and vice versa. The operating means 205 may operate the control valves Vhi3, Vhi4, namely the purge electrically-operated valves, so that they are both in the closed position so as to prevent, i.e. prohibit or halt, any circulation of the gas in the rest of the purge circuit 250, namely towards the purge outlet 6.

Furthermore, the apparatus 1 also comprises purge initiation means 7 that can be actuated, i.e. activated, by the user in order to begin a purge procedure, namely to indicate to the operating means 205 that it is necessary to control the first and second flow control means 210, 252, typically the main electrically-operated valves Vhi1, Vhi2 and secondary electrically-operated valves Vhi3, Vhi4 so as to control the gas flows circulating in the main circuit 200 and the purge circuit 250.

Advantageously, the purge initiation means 7 comprise a virtual, i.e. digital, purge-start button 7.1 displayed on the display touchscreen 4 of the apparatus 1. This virtual purge-start button 7.1 may be “activated” by the user bringing a finger, for example the index finger, into contact with it or pressing on it.

When it is “activated” finger contact with the relevant zone of the touchscreen 4 is detected and an activation signal is transmitted to the operating means 205 which then interpret this as a signal to begin a purge, i.e. to start the purging of the flexible conduit with a view to replacing one of the gas cylinders 10.

In response to the user pressing on the virtual purge-start button 7.1 and to the begin-purge signal, the operating means 205 are configured to operate the main and purge electrically-operated valves Vhi1, Vhi2; Vhi3, Vhi4 in such a way as to:
halt the circulation of gas in that part of the main gas circuit 200 that is situated downstream of the main electrically-operated valves Vhi1, Vhi2, namely in part of the inlet portions 201, by closing, or keeping closed, the two main electrically-operated valves Vhi1, Vhi2.
keep one of the purge electrically-operated valves (Vhi3, Vhi4) closed and
allow gas to circulate in the purge circuit (250) by moving the other of the purge electrically-operated valves (Vhi3, Vhi4) into the open position in order to place the corresponding purge portion 251, the inlet portion 201 fluidically connected to it, and the flexible conduit 12 that is coupled to the latter, in fluidic communication with one another and also with the atmosphere, via the purge orifice 6, so as to purge the gas pressure present in the flexible conduit 12 and in the inlet portion 201 of the main circuit 200 and thus allow the user to then, without risk, disconnect the flexible conduit 12 from the pressure regulator device 102 and the latter from the cylinder 10 that is to be replaced because it is empty or almost empty.

According to one particular embodiment, the operating means 205 are configured to operate the main and purge electrically-operated valves Vhi1, Vhi2; Vhi3, Vhi4 and commence a purge after a first press on the purge-start button 7.1, followed by a second press on a virtual validation button (not shown) displayed on the screen 4, i.e. a selection-confirmation button.

These virtual start-purge and validation buttons may be displayed inside a window displayed on the screen 4 of the apparatus.

According to one embodiment, the NO cylinders 10 and/or the NO delivery apparatus 1 may be arranged on a transport cart (not shown) like the one described by EP4159585 or EP4461619.

In general terms, i.e. not restricted to the embodiment of the figures, the invention also relates to a method for replacing an (almost) empty NO vessel, hereinafter referred to as an (almost) empty NO cylinder 10 fluidically coupled by a flexible conduit or hose 12 to the main gas circuit 200 of the NO delivery apparatus 1 of a gas administration installation 100 according to the invention, such as that of FIG. 1, with a full NO cylinder (i.e. a “new” vessel), said flexible conduit 12 containing gaseous NO, typically an NO/N₂ mixture at a residual pressure greater than 1 bar, wherein:
a) a procedure of purging said flexible conduit 12 is begun by a user pressing a finger (or another digit) on a virtual purge-start button 7.1 displayed on the display touchscreen 4 of the NO delivery apparatus 1, so as to generate (i.e. through the pressing of the finger) an activation signal that is transmitted to the operating means 205 of the NO delivery apparatus 1,
b) in response to said activation signal, the operating means 205 of the NO delivery apparatus 1 control the flows of gas, i.e. of gaseous NO, circulating in the main circuit 200 and the purge circuit 250 of the NO delivery apparatus 1, so as to:
   halt the circulation of gas in that part of the main gas circuit 200 that is situated downstream of at least one main electrically-operated valve Vhi1, Vhi2 arranged on said main gas circuit 200 of the NO delivery apparatus 1 by closing, or keeping closed, said at least one main electrically-operated valve Vhi1, Vhi2, and
   allow gas to circulate in the purge circuit 250 of the NO delivery apparatus 1 by moving a (first) purge electrically-operated valve Vhi3, Vhi4 into the open position in order to place that portion of the main gas circuit 200 that is fluidically connected to the flexible conduit 12 that is to be purged in fluidic communication with the atmosphere, i.e. via a purge orifice 6 so as to discharge into the atmosphere at least some of the gaseous NO at the residual pressure that is present in the flexible conduit 12 that is to be purged and in said portion of the main circuit 200,
c) the user disconnects (i.e. detaches) the flexible conduit 12 from the empty cylinder 10 that is to be replaced and/or from a pressure regulator device 102 arranged between the flexible conduit 12 and the empty cylinder 10 that is to be replaced, and
d) the user (re)connects (i.e. re-attaches) the flexible conduit 12 to a full cylinder 10 i.e. replacement “new” vessel, and/or to the pressure regulator device 102 arranged between the flexible conduit 12 and the full cylinder 10.

In steps c) and d), according to the embodiment, the flexible conduit 12 may be fluidically connected/coupled:
directly to the NO cylinder 10, typically to a valve (also known as a tap or the like) with which the gas cylinder is equipped and which includes internal gas pressure reducing means, namely a valve with an integrated pressure regulator or IGPR (not illustrated), allowing the gas pressure to be reduced and a desired pressure, for example of under 10 bar, to be supplied; or
indirectly to the NO cylinder 10, by coupling it to an external pressure regulator device 102, itself arranged between the flexible conduit 12 and the gas cylinder 10, typically attached to the valve fitted to the gas cylinder, as illustrated in FIG. 1. In that case, it is the pressure regulator device 102 that reduces the pressure of the gas and allows it to be supplied at the desired pressure, e.g. less than 10 bar.

Depending on the embodiment under consideration, the method of the invention may comprise one or more of the following features:
it further comprises keeping a second purge electrically-operated valve Vhi3, that forms part of the purge electrically-operated valves Vhi3, Vhi4, closed when the first purge electrically-operated valve Vhi4, i.e. the other purge electrically-operated valve, is in the open position.
in step a) the user's pressing of the virtual purge-start button 7.1 with a finger (or other digit) needs to be followed by a second pressing of a virtual validation button (i.e. a selection confirmation button) displayed on the screen 4 in order to generate the activation signal.
when replacing an empty cylinder with a full cylinder, in order to avoid any interruption to the treatment of the patient, another full NO cylinder takes over to supply the NO/N₂ mixture to the NO delivery apparatus, particularly at a part of the main circuit 200 that is not in fluidic communication with the main gas circuit portion fluidically connected to the atmosphere via the purge orifice 6.
the main electrically-operated valves Vhi1, Vhi2 and secondary electrically-operated valves Vhi3, Vhi4 form part of the flow control means 210, 252 of the NO delivery apparatus 1.
in response to the user pressing on the virtual purge-start button 7.1 and to the begin-purge signal, the operating means 205 are configured to operate the main and purge electrically-operated valves Vhi1, Vhi2; Vhi3, Vhi4.
a flexible hose 12 to the main gas circuit 200 of the NO delivery apparatus 1.
two NO cylinders 10 are each fluidically coupled by a flexible hose 12 to the main gas circuit 200 of the NO delivery apparatus 1.
when one of the NO cylinders 1 is empty and in the process of being replaced, the other supplies the main gas circuit 200 of the NO delivery apparatus 1 with a flow of gaseous NO, i.e. with an NO/N₂ mixture.
the NO cylinder(s) 10 contain a gaseous NO/N₂ mixture.
the NO cylinder(s) 10 are equipped with a gas distribution valve 101.
a gas pressure regulator device is attached to the gas distribution valve 101 of the NO cylinder(s) 10.
each flexible hose is fluidically coupled to a gas pressure regulator device.
replacing an empty cylinder with a full cylinder comprises the operations of:
   disconnecting the flexible hose or conduit from the pressure regulator device,
   detaching, notably unscrewing, the pressure regulator device from the empty cylinder, typically from the valve with which the empty cylinder is equipped,
   reattaching, notably by screw fastening, the pressure regulator device to the replacement full NO cylinder, typically to the valve with which the full cylinder is equipped, and
   fluidically (re-)connecting the flexible hose or conduit to the pressure regulator device.
the NO cylinders 10 and/or the NO delivery apparatus 1 are arranged on a transport cart.

In general, a gas administration installation 100 according to the invention can be used to administer nitric oxide (NO), i.e. the final NO/O₂/N₂ mixture obtained, by inhalation to persons, i.e. patients, suffering from acute pulmonary hypertension, in particular to dilate their pulmonary vessels and increase their oxygenation by improving pulmonary gas exchange, in particular to treat persistent pulmonary arterial hypertension of the newborn (PPHN), acute respiratory distress syndrome (ARDS) observed mainly in adults, or pulmonary hypertension (PH) in cardiac surgery, in adults or children.

What is claimed is:

1. NO delivery apparatus (1) for supplying an NO-containing gas, comprising:
a main gas circuit (200) for conveying the NO-containing gas between at least one NO inlet (2) and at least one NO outlet (5),
said at least one NO inlet (2) configured to allow the fluidic connection thereto of a flexible conduit (12) so as to supply the main gas circuit (200) with the NO-containing gas carried by said flexible conduit (12),
first flow-control means (210) arranged on said main gas circuit (200) and configured to control the circulation of the flow of NO-containing gas in said main gas circuit (200),
microprocessor-containing operating means (205),
a purge circuit (250) fluidically coupled (at 253) to the main gas circuit (200) between said at least one NO inlet (2) and the first flow control means (210), said purge circuit (250) comprising a purge outlet (6) in fluidic communication with the atmosphere, and
second flow control means (252) arranged on said purge circuit (250) and configured to control the circulation of gas in said purge circuit (250) towards the purge outlet (6), and
purge initiation means (7) that can be actuated by the user,
characterized in that:
it further comprises a display touchscreen (4),
the purge initiation means (7) comprise a virtual purge-start button (7.1) displayed on the display touchscreen (4), and
the operating means (205) are configured to operate the first and second flow-control means (210; 252) in such a way as to halt the circulation of gas in at least part of the main gas circuit (200) and allow gas to circulate in the purge circuit (250) in response to the user pressing a finger on the virtual button (7.1).

2. Apparatus according to claim 1, characterized in that the first and second flow control means (210; 252) comprise at least one valve (Vhi1, Vhi2; Vhi3, Vhi4).

3. Apparatus according to claim 1, characterized in that:
the purge-start button (7.1) is configured to be activated by the user bringing a finger into contact with or pressing on a zone of the touchscreen (4),
the touchscreen (4) is configured to detect contact of the user's finger on the zone of the touchscreen (4) that corresponds to the purge-start button (7.1) when the purge-start button (7.1) is activated, and to transmit an activation signal to the operating means (205), and
said operating means (205) are configured to interpret this activation signal as a signal to begin the purge.

4. Apparatus according to claim 1, characterized in that the operating means (205) command a purge to begin by acting on the first and second flow control means (210; 252) in response to a finger pressing on the virtual button (7.1) and/or to the activation signal.

5. Apparatus according to claim 1, characterized in that:
the main gas circuit (200) comprises two inlet portions (201) arranged in parallel, each inlet portion (201) comprising an NO inlet (2), said two inlet portions (201) being fluidically coupled to one another at a junction point (202) of the main gas circuit (200); and
the purge circuit (250) comprises two joining portions (251) arranged in parallel, each joining portion (251) being coupled (at 253) to one of the inlet portions (201) of the main gas circuit (200), upstream of the first flow control means (210) arranged on said main gas circuit (200), and said two joining portions (251) are fluidically coupled to one another at a meeting point (254) of the purge circuit (250).

6. Apparatus according to claim 5, characterized in that second flow control means (252) are arranged on the two joining portions (251).

7. Apparatus according to claim 1, characterized in that, in response to the user pressing on the virtual purge-start button (7.1) displayed on the display touchscreen (4), the operating means (205) are configured to operate the main and purge electrically-operated valves (Vhi1, Vhi2; Vhi3, Vhi4) in such a way as to:
halt the circulation of gas in that part of the main gas circuit (200) that is situated downstream of the main electrically-operated valves (Vhi1, Vhi2) by closing, or keeping closed, the two main electrically-operated valves (Vhi1, Vhi2),
keep one of the purge electrically-operated valves (Vhi3, Vhi4) closed and
allow gas to circulate in the purge circuit (250) by moving the other of the purge electrically-operated valves (Vhi3, Vhi4) into the open position in order to allow the gas to circulate in the purge circuit (250).

8. Apparatus according to claim 1, characterized in that the operating means (205) comprise a microcontroller.

9. Apparatus according to claim 1, characterized in that the first and second flow control means (210; 252) comprise electrically-operated valves (Vhi1, Vhi2; Vhi3, Vhi4) operated by the operating means (205).

10. Installation (100) for supplying an NO-containing gas mixture to a patient, comprising:
at least one NO vessel (10) containing the NO-containing gas, preferably two vessels (10) for NO, typically an NO/N₂ gas mixture,
the NO delivery apparatus (1) according to claim 1; and
at least one flexible conduit (12) fluidically connecting said at least one NO vessel (10) to the NO delivery apparatus (1), said at least one NO vessel (10) and/or the NO delivery apparatus (1) preferably being arranged on a transport cart.

11. Installation (100) according to claim 10, characterized in that at least one pressure-reducing device (102) is arranged downstream of said at least one NO vessel (10), said at least one flexible conduit (12) being fluidically tuned to said at least one pressure-reducing device (102).

12. Installation according to claim 10, characterized in that it further comprises a medical ventilator (50) configured to supply a flow of O₂-containing respiratory gas; and a respiratory circuit (20; 21) to which the medical ventilator (50) and the NO delivery apparatus (1) are fluidically coupled.

13. Installation according to claim 10, characterized in that each NO vessel (10) contains an NO/N₂ gaseous mixture containing between 100 and 2000 ppmv of NO, the rest being nitrogen (N₂) and/or each pressure-reducing device (102) is configured to reduce the pressure of the NO/N₂ gas mixture to an expansion pressure of less than or equal to 10 bar, typically of between 2 and 7 bar.

14. Installation according to claim 1, characterized in that the NO delivery apparatus (1) comprises two NO inlets (2) configured each to be coupled to one flexible conduit (12) for conveying the NO/N₂ mixture.

15. Method for replacing an empty NO vessel (10) fluidically coupled by a flexible conduit (12) to the main gas circuit (200) of the NO delivery apparatus (1) and/or of a gas administration installation (100) according to Claim 10 with a full NO vessel (10), said flexible conduit (12) containing gaseous NO, typically an NO/N₂ mixture at a residual pressure greater than 1 bar, typically a residual pressure of under 10 bar, wherein:
a. a procedure of purging said flexible conduit (12) is begun by a user pressing a finger on a virtual purge-start button (7.1) displayed on the display touchscreen (4) of the NO delivery apparatus (1), so as to generate an activation signal that is transmitted to the operating means (205) of the NO delivery apparatus (1),
b. in response to said activation signal, the operating means (205) of the NO delivery apparatus (1) control the flows of gas circulating in the main circuit (200) and the purge circuit (250) of the NO delivery apparatus (1), so as to:
halt the circulation of gas in that part of the main gas circuit (200) that is situated downstream of at least one main electrically-operated valve (Vhi1, Vhi2) arranged on said main gas circuit (200) of the NO delivery apparatus (1) by closing, or keeping closed, said at least one main electrically-operated valve (Vhi1, Vhi2), and
allow gas to circulate in the purge circuit (250) of the NO delivery apparatus (1) by moving a purge electrically-operated valve (Vhi4, Vhi3) into the open position in order to place that portion of the main gas circuit (200) that is fluidically connected to the flexible conduit (12) that is to be purged in fluidic communication with the atmosphere, preferably via a purge orifice (6) so as to discharge into the atmosphere at least some of the gaseous NO at the residual pressure that is present in the flexible conduit (12) that is to be purged and in said portion of the main circuit (200),
c. the user disconnects the flexible conduit (12) from the empty NO vessel (10) that is to be replaced and/or from a regulator device (102) arranged between the flexible conduit (12) and the empty NO vessel (10) that is to be replaced, and
d. the user connects the flexible conduit (12) to a full NO vessel (10) and/or to the regulator device (102) arranged between the flexible conduit (12) and said full NO vessel (10).
